(19) United States
(12) Patent Application Publication
(10) Pub. No.: US 2023/0321175 A1
(43) Pub. Date: Oct. 12, 2023

(54) ANTI-DIABETIC STEROIDAL LACTONES OF WITHANIA COAGULANS FOR TREATMENT OF TYPE 2 DIABETES

(71) Applicants: Khalifa University of Science and Technology, Abu Dhabi (AE); United Arab Emirates University, Al Ain (AE)

(72) Inventors: Abdu ADEM, Abu Dhabi (AE); Mariyam KHALID, Al Ain (AE); Juma AL KAABI, Al Ain (AE); Naheed AMIR, Al Ain (AE); Samir ATTOUB, Al Ain (AE)

(21) Appl. No.: 17/716,189

(22) Filed: Apr. 8, 2022

(51) Int. Cl. A61K 36/81; A61P 43/00; A61K 31/215; A61P 21/00; A61K 38/28; A61K 38/01

(52) U.S. Cl. CPC A61K 36/81; A61P 43/00; A61K 31/215; A61P 21/00; A61K 38/28; A61K 38/012

(57) ABSTRACT

Embodiments of the present disclosure include compositions and methods for treating type 2 diabetes using anti-diabetic steroidal lactones of Withania Coagulans, such as Coagulansin A and derivatives and analogues thereof are described. For example, a method of treating type 2 diabetes in a subject in need thereof, the method comprising administering a therapeutically effective amount of Coagulansin A or a derivative or analogue thereof to the subject is provided. Further described embodiments include compositions and methods of treating muscle atrophy in a subject with type 2 diabetes by initiating regeneration of diseased skeletal muscle myoblasts using anti-diabetic steroidal lactones of Withania Coagulans.

### BACKGROUND

Type 2 diabetes is associated with reduced skeletal muscle metabolism and repair, amongst other symptoms. The inability to properly regulate glucose metabolism due to impairment in insulin sensitivity and GLUT4 expression results in reduced skeletal muscle mass, strength, and quality and further exacerbates this systemic disorder. There is a crucial need for the identification of anabolic therapies that are effective in skeletal muscle insulin resistance and can initiate muscle regeneration.

### SUMMARY

In general, the present disclosure features compositions and methods for treating type 2 diabetes using anti-diabetic steroidal lactones of Withania Coagulans, such as Coagulansin A and derivatives and analogues thereof.

In a first aspect, the present disclosure describes a composition for the treatment of Type 2 diabetes, the composition comprising a therapeutically effective amount of Coagulansin A and a pharmaceutically or nutraceutically acceptable carrier, wherein the therapeutically effective amount is effective for treating muscle atrophy, treating insulin resistance in skeletal muscle, initiating muscle regeneration, upregulating myogenic regulatory factors, repressing atrogin-1 expression, improving insulin-stimulated glucose uptake, inhibiting advanced glycation end-products (AGEs)-mediated β-cell death, increasing β-cell density, enhancing pancreatic islets volume, upregulating insulin-dependent glucose transporter GLUT4 expression in skeletal muscle, or a combination thereof. The composition can be formulated for buccal, sublingual, oral, nasal, pulmonary, transdermal, intravenous, intraarterial, intraperitoneal, intramuscular, or subcutaneous administration. The composition can be formulated as a capsule, tablet, pill, dragee, powder, granule, emulsion, solution, suspension, syrup, elixir, or implant. The composition can be formulated for delayed release, extended release, or immediate release. The composition include one or more other active pharmaceutical ingredients for the treatment of Type 2 diabetes, such as insulin, an Alpha-glucosidase inhibitor, a biguanide a dopamine agonist, a Dipeptidyl peptidase-4 (DPP-4) inhibitor, a Glucagon-like peptide-1 receptor agonist, a meglitinide, a Sodium-glucose transporter (SGLT) 2 inhibitor, a sulfonylurea, a thiazolidinedione, or a combination thereof.

In a second aspect, the present disclosure describes a method of treating type 2 diabetes in a subject in need thereof, the method comprising administering a therapeutically effective amount of an aqueous extract of Withania Coagulans fruit, wherein the aqueous extract comprises Coagulansin A, or a derivative or analogue thereof. The therapeutically effective amount is an amount that is effective for treating muscle atrophy, treating insulin resistance in skeletal muscle, initiating muscle regeneration, upregulating myogenic regulatory factors, repressing atrogin-1 expression, improving insulin-stimulated glucose uptake, inhibiting advanced glycation end-products (AGEs)-mediated β-cell death, increasing β-cell density, enhancing pancreatic islet volume, upregulating insulin-dependent glucose transporter GLUT4 expression in skeletal muscle, or a combination thereof. The extract can be administered in combination with insulin, an Alpha-glucosidase inhibitor, a biguanide, a dopamine agonist, a Dipeptidyl peptidase-4 (DPP-4) inhibitor, a Glucagon-like peptide-1 receptor agonist, a meglitinide, a Sodium-glucose transporter (SGLT) 2 inhibitor, a sulfonylurea, a thiazolidinedione, or a combination thereof. The extract can be administered via a buccal, sublingual, oral, nasal, pulmonary, transdermal, intravenous, intraarterial, intraperitoneal, intramuscular, or subcutaneous route. The subject can be a mammal. In some cases, the mammal is a human. The extract can be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg extract, of subject body weight per unit dose. The extract can be administered at a dosage level sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg Coagulansin A, of subject body weight per unit dose. The extract can be administered one or more times a day. The extract can be administered at least once a day for at least two days. The extract can be administered with at least one meal, optionally at the start of the meal, or after the meal. The extract can be administered as an adjunct to diet and exercise to improve glycemic control.

In another aspect, the present disclosure describes a first medical use of Coagulansin A as a medicament.

In another aspect, the present disclosure describes a further medical use of an aqueous extract of Withania Coagulans fruit for treatment of Type 2 diabetes. The aqueous extract can include a therapeutically effective amount of Coagulansin A, or a derivative or analogue thereof. In some cases, the therapeutically effective amount is the amount that is effective for treating muscle atrophy, treating insulin resistance in skeletal muscle, initiating muscle regeneration, upregulating myogenic regulatory factors, repressing atrogin-1 expression, improving insulin-stimulated glucose uptake, inhibiting advanced glycation end-products (AGEs)-mediated β-cell death, increasing β-cell density, enhancing pancreatic islets volume, upregulating insulin-dependent glucose transporter GLUT4 expression in skeletal muscle, or a combination thereof.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. The drawings illustrate generally, by way of example, but not by way of limitation, various embodiments discussed in the present document. The application file contains at least one drawing executed in color, copies of which can be provided by the Office upon request and payment of the necessary fee

Reference is made to illustrative embodiments that are depicted in the figures, in which:

FIG. 1 is a schematic representation of the experimental design of diseased human skeletal muscle myoblasts (D-HSMM), according to one or more embodiments of the present disclosure.

FIGS. 2A-B show graphical representations of the effect of an aqueous extract of W. Coagulans fruit (hereafter “W. Coagulans”) and Coagulansin-A on LDH release (A) and MTT (B), in type 2 diabetes diseased human skeletal muscle myoblasts (D-HSMM) myoblasts treated for 48 hrs. Insulin, metformin, glibenclamide, and pioglitazone were used as the positive control. Data are expressed as a percentage of control. Bars represent Mean±SEM, of 3 determinations (n=3). **p<0.01, *p<0.05 vs. control (non-treated cells).

FIGS. 3A-B show the effect of W. Coagulans and Coagulansin A on myogenic regulatory factors. Histogram presenting myogenin fluorescence intensity (A), representative images of immunofluorescence staining of myogenin (red) and DAPI (blue) in D-HSMM at a final magnification of 200×(B). Insulin and metformin are used as a positive control. Data are expressed as a percentage of control. Bars represent Mean±SEM, of 3 determinations (n=3). **p<0.01, *p<0.05 vs. control (non-treated cells); ⋄p<0.05 vs. metformin treated cells.

FIGS. 4A-B show the effect of W. Coagulans and Coagulansin-A on myogenic regulatory factors. Histogram presenting atrogin1 fluorescence intensity (A), representative images of immunofluorescence staining of atrogin1 (green) and DAPI (blue) in D-HSMM at a final magnification of 200×(B). Insulin and metformin are used as a positive control. Data are expressed as a percentage of control. Bars represent Mean±SEM, of 3 determinations (n=3). ***p<0.001 vs. control (non-treated cells).

FIGS. 5A-B show the effect of W. Coagulans and Coagulansin-A on the regulation of GLUT4. Histogram presenting MyoD positive cells (A), representative images of immunofluorescence staining of MyoD (red) and DAPI (blue) in D-HSMM at a final magnification of 200×(B). Insulin, metformin, glibenclamide, and pioglitazone were used as the positive control. Data are expressed as a percentage of control. Bars represent Mean±SEM, of 3 determinations (n=3). ***p<0.001, **p<0.01, *p<0.05 vs. control (non-treated cells); γγp<0.01 vs. metformin treated cells; γp<0.01 vs. pioglitazone treated cells.

FIGS. 6A-B shows the effect of W. Coagulans and Coagulansin-A on GLUT1 (A), GLUT4 (B) in D-HSMM myotubes. Insulin, metformin, glibenclamide, and pioglitazone are used as the positive control. Data are expressed as a percentage of control. Bars represent Mean±SEM, of 3 determinations (n=3). **p<0.01 vs. control (non-treated cells); γγp<0.01, γp<0.05 vs. pioglitazone treated cells.

FIG. 7 shows the effect of W. Coagulans and Coagulansin-A on glucose uptake. Insulin-stimulated 2-NBDG uptake in D-HSMM myotubes. Insulin, metformin, glibenclamide, and pioglitazone are used as the positive control. Data are expressed as a percentage of control. Bars represent Mean±SEM, of 3 determinations (n=3). **p<0.01 vs. control (non-treated cells); ##p<0.01 vs. insulin treated cells; ⋄p<0.05 vs. metformin treated cells; γγp<0.01 vs. pioglitazone treated cells.

FIGS. 8A-B show the effect of W. Coagulans and Coagulansin-A on LDH release (A), MTT (B), in human type 2 diabetic skeletal muscle myoblasts (D-HSMM) myoblasts treated for 48 hrs. Data are expressed as a percentage of control. Bars represent Mean±SEM, of 3 determinations (n=3). **p<0.01, *p<0.05 vs. control (non-treated cells).

FIG. 9 shows dose optimization of W. Coagulans and Coagulansin-A. Histogram presenting MyoD positive cells. Data are expressed as a percentage of control. Bars represent Mean±SEM, of 3 determinations (n=3). **p<0.01, *p<0.05 vs. control (non-treated cells).

FIGS. 10A-C describe the in vivo effect of Withania Coagulans fruit extract administration on streptozotocin-induced eight months chronic diabetic rats on: (A) fasting blood glucose levels; (B) serum insulin levels; and (C) serum glucagon levels. (NC: non-diabetic control; NWC: non-diabetic Withania Coagulans treated; DC: Diabetic control; DWC: Diabetic Withania Coagulans treated).

FIGS. 11A-D describe the positive in vivo effect of Withania Coagulans fruit extract administration on: (A) serum Advanced Glycation End-products (AGEs) levels; (B) pancreatic AGEs levels; (C) pancreatic Receptor AGEs (RAGE) expression levels compared to the diabetic control group; and (D) pancreatic islet amyloid polypeptide (IAPP).

FIG. 12A-C describes the protective effect of Withania Coagulans fruit extract administration on: (A) Superoxide Dismutase (SOD) activity; (B) Catalase activity; and (C) glutathione (GSH) levels.

FIG. 13A-C describes the mitigation of pancreatic malondialdehyde (MDA), nitric oxide (NO), and C-reactive protein (CRP) after Withania Coagulans fruit extract administration.

FIGS. 14A-B show immunofluorescence analysis (A) and imaging (B) of IAPP and Insulin expression in pancreatic Beta Cells.

FIGS. 15A-C show immunofluorescence of analysis of Pancreatic beta cell growth (A) and Islet cell viability (B); (C) shows immunofluorescence insulin and DNA (DAPI).

### DETAILED DESCRIPTION

In general, embodiments of the present disclosure include compositions and methods of treating type 2 diabetes using anti-diabetic steroidal lactones of Withania Coagulans, such as Coagulansin A and derivatives and analogues thereof.

### Definitions

The terms recited below have been defined as described below. All other terms and phrases in this disclosure shall be construed according to their ordinary meaning as understood by one of skill in the art.

As used herein, “Coagulansin-A” refers to the steroidal lactone from W. coagulans having the following structure:

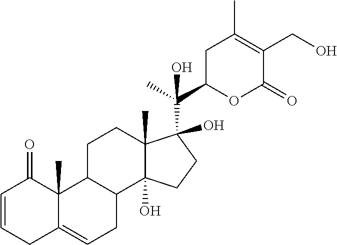 [SMILES: CC1=C(CO)C(=O)O[C@@H]([C@](C)(O)[C@]2(O)CC[C@@]3(O)C4CC=C5CC=CC(=O)[C@]5(C)C4CC[C@]23C)C1]

(14α,17S,20S,22R)-14,17,20,27-tetrahydroxy-1-oxowitha-2,5,24-trienolide).

Lactones are cyclic carboxylic esters, containing a 1-oxacycloalkan-2-one structure (—(C═O)—O—), or analogues having unsaturation or heteroatoms replacing one or more carbon atoms of the ring. Steroidal compounds have four rings arranged in a specific molecular configuration and generally includes three six-member cyclohexane rings and one five-member cyclopentane ring. “Steroidal lactones” or “lactone steroids” as used herein refer to compounds having a steroid backbone bound to a lactone or one of its derivatives. Exemplary lactone steroids encompassed by the present disclosure include Coagulansin A, and include the use of synthesized non-natural analogues of Coagulansin A. As used herein, the term “analogue”, “derivative”, or the like is meant to refer to a change or substitution of an atom with another atom or group. For example, when a hydrogen is replaced with a halogen or a hydroxyl group, such a change produces a derivative. A non-natural product is a compound that is artificially produced or synthesized and not found in nature. The term “synthesized” means that the compound is chemically produced (e.g. in a laboratory) as opposed to being isolated from the natural environment if it is naturally occurring. The derivatives and analogues of the anti-diabetic steroidal lactones of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof.

The term “treating” or “treatment”, as used herein, means reversing, alleviating, inhibiting the progress of, or ameliorating the disorder or condition to which such term applies, or one or more symptoms of such disorder or condition. For example, treatment with a therapeutically effective amount of an anti-diabetic steroidal lactone of the disclosure (e.g., Coagulansin A) may reverse, slow or inhibit muscle atrophy associated with Type 2 diabetes e.g. by initiating muscle regeneration and/or upregulation of myogenic regulatory factors (e.g., MyoD and/or myogenin), and/or repression of atrogin-1 expression. In some cases, treatment with a therapeutically effective amount of an anti-diabetic steroidal lactone of the present disclosure (e.g., Coagulansin A) can ameliorate insulin resistance of skeletal muscle associated with Type 2 diabetes by improving insulin-stimulated glucose uptake and/or upregulating insulin-dependent glucose transporter GLUT4 expression in the skeletal muscle of a patient in need thereof. In some embodiments, treatment with a therapeutically effective amount of an anti-diabetic steroidal lactone of the present disclosure (e.g., Coagulansin A) can improve pancreatic β-cells mass and/or function by attenuating oxidative stress induced by Advanced glycation end products (AGEs), and their interaction with receptors therefor (RAGE).

By a “therapeutically effective amount” is meant a sufficient amount of the molecule to treat Type 2 diabetes or a disorder that induces insulin resistance and/or atrophy in skeletal muscle, and/or AGE/RAGE mediated β-cell death, at a reasonable benefit/risk ratio applicable to any medical treatment. It will be understood, however, that the total daily usage of a compositions of the present disclosure will be decided by the attending physician within the scope of sound medical judgment. The specific therapeutically effective dose level for any particular subject will depend upon a variety of factors including the symptoms of the disorder being treated and the severity of the disorder; activity of the steroidal lactone employed; the specific composition employed, the age, body weight, general health, sex and diet of the subject; the time of administration, route of administration, and rate of excretion of the specific steroidal lactone employed; the duration of the treatment; drugs used in combination or coincidental with the specific steroidal lactone employed; and like factors well known in the medical arts. For example, it is well within the skill of the art to start doses of the anti-diabetic steroidal lactone at levels lower than those required to achieve the desired therapeutic effect and to gradually increase the dosage until the desired effect is achieved. A effective dose of a specific anti-diabetic steroidal lactone can be within the range of about 0.01 femtogram (fg)/ml to about 0.1 mg/ml, such as 1 fg/ml, 10 fg/ml, 100 fg/ml, 1 pm/ml, 10 pm/ml, 100, pm/ml, 1 nm/ml, 10 nm/ml, 100 nm/ml, 1 μg/ml, 10 μg/ml, and 100 μg/ml, with all intervening values being within the scope of this disclosure.

“Pharmaceutically” or “pharmaceutically acceptable” refer to compounds (e.g., carriers or other excipients) and compositions that do not produce an adverse, allergic or other untoward reaction when administered to a subject, such as a human, as appropriate. A pharmaceutically acceptable carrier or excipient refers to a non-toxic solid, semi-solid or liquid filler, diluent, encapsulating material or formulation auxiliary of any type.

The term “nutraceutical” (or “nutraceutically acceptable”), as used herein, refers to any food or food ingredient (or additive) considered to provide medical or health benefits, including the prevention or treatment of disease such as type 2 diabetes, or a symptom thereof.

One embodiment of the present disclosure Coagulansin A, or an analogue or derivative thereof, is provided for use as a medicament. In some cases, Coagulansin A is used in the treatment of type 2 diabetes, in the treatment of muscle atrophy, in the treatment of insulin resistance in skeletal muscle, and/or in the treatment of hyperglycemia-induced β-cell damage mediated by advanced glycation end products (AGEs) and/or a receptor therefore (RAGE).

In another embodiment of the present disclosure, the use of an aqueous extract of a fruit of W. coagulans for the treatment of type 2 diabetes is provided. For example, the aqueous extract is used in the treatment of muscle atrophy, in the treatment of insulin resistance in skeletal muscle, and/or in the treatment of hyperglycemia-induced β-cell damage mediated by AGE/RAGE.

Also provided is a method of treatment of type 2 diabetes in a subject in need thereof, comprising administering to the subject a therapeutically effective amount of an anti-diabetic steroidal lactone of Withania coagulans, or a derivative or analogue thereof. The steroidal lactone, derivative or analogue can be naturally produced by a W. coagulans plant, isolated from the plant, or synthesized. The steroidal lactone can be isolated from fruit or other aerial plant part. In one embodiment, the anti-diabetic steroidal lactone is Coagulansin A. In one embodiment, the steroidal lactone is Coagulansin A, isolated from dried fruit by methanolic extraction. The anti-diabetic steroidal lactone can be formulated with a pharmaceutically acceptable carrier or other excipient. In some cases, the anti-diabetic steroidal lactone can be formulated with a nutraceutically acceptable carrier.

The identity and amounts of natural products isolated can vary based on how the plant is grown. For example, when the plant is grown aeroponically, using chemically-defined nutrient media and without soil, natural products can be isolated. In certain embodiments, the amount of a particular natural product may be altered by growing the plant under different conditions. In some cases, the plant material is grown in a specific geographic region and/or under specific conditions to optimize the profile of steroid lactones for a use of the present disclosure.

Any method of administration may be used to deliver the compound of the disclosure to the subject. In particular embodiments, the route of administration may be buccal, sublingual, oral, nasal, pulmonary, transdermal, intravenous, intraarterial, intraperitoneal, intramuscular, or subcutaneous, for example. Thus, solid and liquid dosage forms are within the scope of this disclosure.

The extract or anti-diabetic steroidal lactone can be formulated with a carrier. In some cases, the carrier is a solvent or dispersion medium containing, for example, water, ethanol, polyol (for example, glycerol, propylene glycol, and liquid polyethylene glycol, and the like), suitable mixtures thereof, and vegetables oils. In many cases, it will be preferable to include isotonic agents, for example, sugars or sodium chloride. Prolonged absorption of the injectable compositions can be brought about by the use in the compositions of agents delaying absorption, which are known in the art of pharmaceutics.

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, DMSO, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents, alcohols, oils, modified oils, glycols, polysorbates, cyclodextrins, polymers, and combinations thereof.

Injectable formulations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the extract or steroidal lactone, it may be desirable to slow the absorption of the drug from subcutaneous or intramuscular injection. Delayed absorption of a parenterally administered drug form is accomplished by dissolving or suspending the drug in an oil vehicle. Injectable, or implantable depot forms are made by forming micro-encapsule matrices of the drug in biodegradable polymers such as poly(lactide-co-glycolide). Depending upon the ratio of drug to polymer and the nature of the particular polymer employed, the rate of drug release can be controlled. Examples of other biodegradable polymers. Depot injectable formulations, such as implants, can also prepared by entrapping the drug in liposomes or microemulsions which are compatible with body tissues.

Solid dosage forms for oral administration include capsules, tablets, pills, dragees, powders, and granules. In such solid dosage forms, the active compound is mixed with at least one inert, pharmaceutically acceptable excipient or carrier, fillers or extenders binders, humectants, disintegrating agents, lubricants and mixtures thereof.

Solid compositions may also be employed as fillers in soft and hard-filled gelatin capsules using such excipients. The solid dosage forms can be prepared with coatings and shells to control release the active ingredient (e.g., in a certain part of the intestinal tract, or in a delayed or extended manner).

Dosage forms for topical or transdermal administration include ointments, pastes, creams, lotions, gels, powders, solutions, sprays, inhalants or patches. For example, a transdermal patch may provide controlled delivery of an anti-diabetic steroidal lactone to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the compound of the disclosure may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg of the extract or a specific steroidal lactone (e.g., Coagulansin A), of subject body weight per day, one or more times a day, to obtain the desired therapeutic effect. Multiple administrations may be by the same route or by different routes. The composition is administered with a meal (e.g., before, during or after the meal). In some embodiments, multiple doses, e.g. 2, 3, 4, 5, or more doses are given over a period of time, e.g. over 2, 3, 4, 5, 6, 7, 8, 9, 10, or more days. In some cases, the composition is administered as an adjunct to diet and exercise to improve glycemic control.

In some embodiments, the compositions described herein are administered without any other active agent. In some embodiments, the compositions described herein may be combined with standard-of-care treatments. For example, in some embodiments, the compound of the disclosure may be administered sequentially or concomitantly with one or more medications for type 2 diabetes. For example, insulin, Alpha-glucosidase inhibitors (acarbose, miglitol), biguanides (metformin along or in combination with alogliptin, canagliflozin, dapagliflozin, empagliflozin, glipizide, glyburide, linagliptin, pioglitazone, repaglinide, rosiglitazone, saxagliptin, sitagliptin), Dopamine agonist (e.g., Bromocriptine), Dipeptidyl peptidase-4 (DPP-4) inhibitors (alogliptin, linagliptin, saxagliptin, sitagliptin), Glucagon-like peptide-1 receptor agonists (GLP-1 receptor agonists) (albiglutide, dulaglutide, exenatide, liraglutide, semaglutide), Meglitinides (nateglinide, repaglinide), Sodium-glucose transporter (SGLT) 2 inhibitors (dapagliflozin, canagliflozin, empagliflozin, ertugliflozin), Sulfonylureas (glimepiride, gliclazide, glipizide, glyburide, chlorpropamide, tolazamide, tolbutamide), Thiazolidinediones (rosiglitazone, pioglitazone).

The invention is further described by the following non-limiting examples which further illustrate the invention. The terminology used in the following example is for the purpose of describing particular embodiments only, and is not intended to be limiting, since the scope of the present invention will be limited only by the appended claims.

### Example 1: Anti-Diabetic Activity of Withania Coagulans and its Constituent Coagulansin-A

Diabetes mellitus is the oldest known disease with a history reaching back to ancient times. Diabetes Mellitus is a series of complex metabolic disorders of multiple etiology and is characterized by chronic hyperglycemia resulting from impaired insulin secretion, insulin action, or most often a combination of both. The deficient insulin action results in disturbances in carbohydrate, protein, and fat metabolism which is associated with long-term dysfunction and failure of various organs and microvascular complications. Type 2 diabetes mellitus is characterized by defects in peripheral insulin sensitivity in skeletal muscle, adipose tissue, and liver that leads to progressive loss of pancreatic beta cells. The non-insulin-dependent type 2 diabetes mellitus accounts for 90-95% of total cases with diabetes. Multiple pathological mechanisms are involved in the development of the metabolic disorder, type 2 diabetes mellitus. Epidemiological studies on the prevalence and the incidence of diabetes mellitus have revealed that around the world, the vast majority of people are affected with type 2 diabetes and this number has dramatically increased in the last few decades. The complications associated with diabetes mellitus make this disease more susceptible to morbidity, mortality and increases the incidence of other diseases

Type 2 diabetes mellitus is a serious medical challenge of the 21st century. Persistently elevated glucose concentrations above the physiological range result in the manifestation of diabetes. The peptide hormone insulin released from pancreatic beta cells maintains normal blood glucose levels by regulating whole-body carbohydrate, lipid, and protein metabolism. Insulin performs its function through signal transduction in insulin-responsive tissues, as these tissues play a distinct role in the regulation of metabolic homeostasis. The resistance against insulin hampers its ability to provide normoglycemia which leads to compensatory hyperinsulinemia. Insulin secretion for a prolonged period exhausts pancreatic beta cells and leads to their apoptosis. The critical literature review of previous studies provides ample evidence that insulin resistance is an apparent foremost defect, evident much before the onset of pancreatic beta cells dysfunction and precedes hyperglycemia leading to type 2 diabetes mellitus.

The skeletal muscle is the largest insulin-sensitive organ that accounts for 85% of whole-body glucose uptake and plays a crucial role in maintaining systemic glucose homeostasis. It is the major peripheral site of insulin-stimulated glucose metabolism and plays a critical role in insulin sensitivity through interactive crosstalk with hepatic and adipose tissues. The skeletal muscle is a major factor to determine the quality of life through regulation of glucose homeostasis, muscle strength to perform the physical function, and capacity to recover from illness. Skeletal muscle insulin sensitivity is the major determinant of protein synthesis through activation of the insulin signal transduction. The prolonged uncontrolled diabetes causes a decrease in muscle strength due to negative regulation of muscle growth and differentiation. The diminished skeletal muscle insulin sensitivity activates proteolytic signaling pathways which impair skeletal muscle metabolism. The imbalance between protein synthesis and degradation disrupts signaling pathways involved in muscle growth and repair. The muscle repair and regeneration process of the adult skeletal muscle recapitulate several aspects of developmental myogenesis. Skeletal muscles are the main protein reservoir as they represent approximately 40-50% of total body weight. The perturbations in insulin signaling and glucose homeostasis in type 2 diabetes mellitus disturb the skeletal muscle protein metabolism. The alteration in muscle energy homeostasis results in prolonged activation of pathways involved in muscle catabolism and transcription of atrogenes. These adaptations lead to the atrophy of skeletal muscles.

Natural products especially medicinal plants exhibit a range of compounds with novel pharmacological properties. Coagulansin-A is a steroidal lactone isolated from the methanolic fraction of W. Coagulans. Coagulansin-A has antiurease activity and has beneficial effects on development of bovine embryo. Previous studies have proved the promising euglycemic effect of W. Coagulans, which may involve multiple mechanisms.

The present study was aimed to identify the effects of aqueous extract of Withania Coagulans (W. Coagulans) and the isolated pure compound Coagulansin-A on GLUT4 expression, glucose uptake and, regeneration in type 2 diabetic skeletal muscle myoblasts.

### Methods:

Cell line: The Clonetics® Diseased Human Skeletal Muscle Myoblasts (D-HSMM) diabetes type II, (Cat no: CC-2901) sourced from a 68-year-old Caucasian male donor, were purchased from Lonza (Lonza Bioscience, USA). Further characteristics of D-HSMM are listed in (Table 1).

TABLE 1
Diseased human skeletal muscle myoblasts
(D-HSMM) cell line characteristics.

| Donor | Characteristics |
|---|---|
| Donor history | Diabetes type II |
| Duration of disease | 20 years |
| Age | 68 years. |
| Sex | Male |
| Race | Caucasian |
| Additional Conditions | MI. Stents. Pacemaker. Congestive |
| | heart failure. Poor circulation. Vision |
| | problems related to Diabetes. |
| Virus testing | Not detected |
| Microbial testing | Negative |
| D-HSMM viability | 91% |
| Desmin test | >=60% pass |

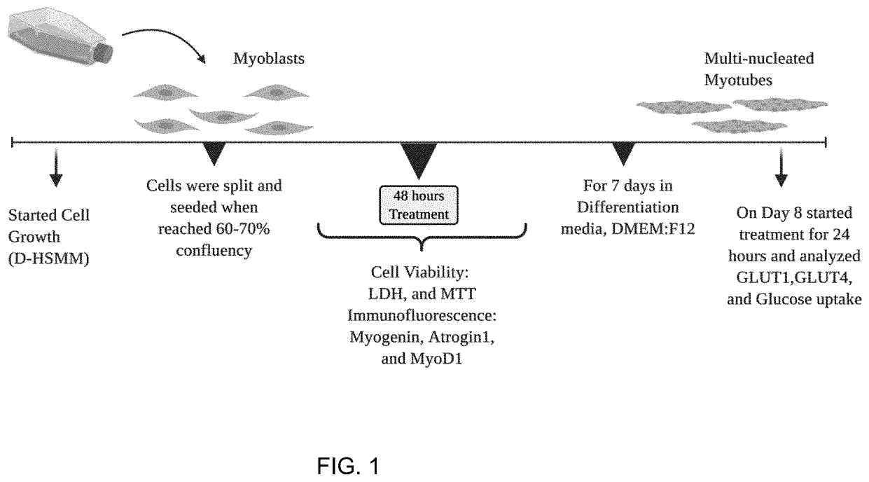

Cells were cultured in the growth medium, Skeletal Muscle Growth Media-2 (SkGM-2; CC-3244) containing basal medium, Human epidermal growth factor (hEGF), Dexamethasone, L-glutamine, Fetal Bovine Serum (FBS), and Gentamicin/Amphotericin B (GA-1000). Cells were seeded at 6250 cells/cm2 in T175 flasks and maintained at 37° C. in a humidified incubator (Thermo Scientific, USA) containing 5% carbon dioxide (CO₂). After 24 hours, when cells adhered to the bottom of the flask, media was changed every 24-48 hours. When cells reached 60-70% confluency, they were trypsinized according to the manufacturer's instructions (Lonza Bioscience, USA), and seeded into appropriate plates according to the requirements for individual assessment (Corning Cellgro, USA). All the experiments were performed at passage number 4-5. The summary of the experimental design is explained in (FIG. 1).

### Preparation of Drug Treatments:

W. Coagulans fruit powder (100 mg/ml) was soaked in distilled water overnight followed by filtration through a 40 μm cell strainer (BD, USA). Working dilutions were prepared in SkGM-2 medium or DMEM:F12 medium from freshly prepared W. Coagulans stock for each experiment. Coagulansin A was provided by Dr. Ihsan-ul-Haq, Quaid-i-Azam University, Pakistan. The stock solution was prepared by dissolving Coagulansin-A (100 mg/ml) in DMSO. All the following dilutions were prepared in SkGM-2 medium or DMEM:F12 medium. The final concentration of DMSO was 10⁻¹²% only in an optimal working concentration.

Insulin (100 nM/6×10⁻⁷ g/ml) (Cat no: 19278, Sigma Aldrich, USA), Metformin (1 nM/2×10⁻² g/ml) (Cat no: PHR1084, Sigma Aldrich, USA), Glibenclamide (10 μM/4×10⁻⁶ g/ml) (Cat no: G0639, Sigma Aldrich, USA), and Pioglitazone (10 μM/5×10⁻⁶ g/ml) (Cat no: E6910, Sigma Aldrich, USA) were used as a positive control. W. Coagulans (1×10⁻¹⁵ g/ml) and Coagulansin-A (1×10⁻¹⁵ g/ml) were used as test drugs.

### Measurement of Cellular Toxicity:

The cytotoxicity detection kit (Cat no: 11644793001, Roche, USA) was used to measure lactate dehydrogenase (LDH) release according to the manufacturer's instructions. Briefly, released LDH in culture supernatants was measured with a 30-mins coupled enzymatic assay that results in the reduction of tetrazolium salt, Iodotetrazolium chloride (INT) into a formazan (red). The amount of color formed was proportional to the number of lysed cells. 2000 Cells were seeded and treated with the optimum concentration of W. Coagulans, Coagulansin-A, insulin, metformin, glibenclamide, and pioglitazone in the SkGM-2 Medium for 48 h in a 96 well, poly D lysine (5 μg/cm2) coated polystyrene plate. Then, 50 μl of cell culture medium was collected from each well and plated into a new microtiter plate. Next, 50 μl of substrate solution was added to the wells, and the plates were incubated for 30 min at room temperature. Absorbance at 490 nm was measured with an Emax Plus microplate reader (CA 94089, Molecular devices, USA). The reader was calibrated to zero absorbance using a culture medium without cells. The untreated cells served as the control, and results were calculated as the percentage of viability in relation to the control.

The percentage of cytotoxicity was calculated as the ratio of experimental values compared with controls as follows:

% LDH release=(A490 test/A490 Control)×100.

Measurement of cellular viability: Cell proliferation was measured by using MTT 3-(4,5-dimethylthiazol-2-yl)-2,5-diphenyltetrazolium bromide (MTT) (Cat no: M2128, Sigma Aldrich, USA). 2000 cells were seeded and treated with the optimum concentration of W. Coagulans, Coagulansin-A, insulin, metformin, glibenclamide, and pioglitazone in the SkGM-2 medium for 48 h in a 96 well (corning, USA) poly D lysine coated polystyrene plate. After 48 h of incubation, plates were removed from the CO₂ incubator and washed with 200 μl PBS. Thereafter, 100 μl of MTT solution (0.5 mg/ml in SkGM-2 medium) was added to each well. The contents were placed on a shaker for 5 min and were incubated for 4 hours in a CO₂ incubator to allow the MTT to be metabolized. After incubation, the supernatant was removed, and the plate was washed with 300 μl PBS. The plates were inverted and strongly tapped on a paper towel to remove the medium completely. The formazan crystals (MTT byproduct) were suspended in 100 ml DMSO and reading was measured at a wavelength of 570 nm by using an Emax Plus microplate reader (Molecular devices, CA 94089, USA). For the blank control, wells were set without cells but received all the reagents. The untreated cells served as the control, and results were calculated as the percentage of viability in relation to the control

### Immunofluorescence Staining

MyoD1: Approximately 2×10³ cells were treated with the optimum concentration of W. Coagulans, Coagulansin-A, insulin, metformin, glibenclamide, and pioglitazone in the SkGM-2 medium for 48 hours in a 96 well black, clear-bottom poly D lysine coated plate (Corning, USA). The supernatant was removed, cells were washed with warm PBS (37° C.), fixed, and permeabilized with 100 μl phosphate-buffered saline (PBS) containing 4% paraformaldehyde and 0.1% Triton X-100 at room temperature for 10 mins. Next, cells were blocked with 1% BSA (100 μl) in PBS at room temperature for 1 hour. The cells were then incubated with 50 μl of primary anti-MyoD1 antibody (Cat no: ab16148, Abcam, USA), diluted 1:100 in a block solution, overnight at 4° C. Cells were washed in PBS three times, incubated with 50 μl Goat anti-Mouse IgG (H+L) secondary antibody (Cross adsorbed, Alexa Fluor 594, Cat no: R37121, Thermo Fisher Scientific, USA) at room temperature for 30 min Nuclei were counterstained with 1 μg/ml Hoechst 33342 solution (Cat no: 62249, Thermo Fisher Scientific, USA) in PBS (100 μl/well).

Myogenin: Approximately 2×10³ cells were treated with the optimum concentration of W. Coagulans, Coagulansin-A, insulin, and metformin in SkGM-2 medium for 48 hours in a 96 well black, clear-bottom poly D lysine coated plate (Corning, USA). The supernatant was removed, cells were washed with warm PBS (37° C.), fixed with 100 μl PBS containing 4% paraformaldehyde at room temperature for 10 mins. Cells were permeabilized with 100 μl of 0.25% Triton X-100 on PBS for 10 mins at 25° C. The cells were then blocked with 100 μl of 10% normal goat serum (NGS), incubated with 50 μl of primary anti-myogenin antibody (Cat no: MAB3876, Merck Millipore, USA) diluted 1:500 in block solution overnight at 4° C. Cells were washed in PBS (200 μl) three times, incubated with 50 μl Goat anti-Mouse IgG (H+L) secondary antibody (Cross adsorbed, Alexa Fluor 594, Cat no: R37121, Thermo Fisher Scientific, USA) at room temperature for 30 min. Nuclei were counterstained with 1 ug/ml Hoechst 33342 solution (Cat no: 62249, Thermo Fisher Scientific, USA) in PBS (100 μl/well).

Atrogin-1/Fbx32: Approximately 2×10³ cells were treated with the optimum concentration of W. Coagulans, Coagulansin-A, insulin, and metformin in SkGM-2 medium for 48 hours in a 96 well black, clear-bottom poly D lysine coated plate (Corning, USA). The supernatant was removed, cells were washed with warm PBS (37° C.), fixed with phosphate-buffered saline (PBS) containing 4% paraformaldehyde at room temperature for 10 mins. Cells were permeabilized with 0.25% Triton X-100 on PBS for 10 mins at 25° C. The cells were then blocked with 100 μl of 10% NGS in PBS at room temperature for 2 hours. The cells were then incubated with 50 μl primary Anti-Fbx32 antibody (Cat no: NBP2-76836, Novus Biological, USA) diluted 1:100 in block solution overnight at 4° C. Cells were washed in PBS three times (200 μl), incubated with 50 μl Goat anti-Rabbit IgG (H+L) (Cross adsorbed, Alexa Fluor 488, Cat no: R37116, Thermo Fisher Scientific, USA) secondary antibody at room temperature for 30 min. Nuclei were counterstained with 1 μg/ml Hoechst 33342 solution (Cat no: 62249, Thermo Fisher Scientific, USA) in PBS (100 μl/well).

For each treatment, cells were examined under EVOS Fl Microscope (Cat no: AMF4300, Life Technologies, USA) and photographed. Approximately 10-12 digital photographs were taken of each treatment for subsequent offline analysis. The total number of immune-positive and immuno-negative cells were counted using Image J (NIH, USA) software. No specific immunostaining was observed in negative control cells. Data were presented as a percentage of control (untreated cells).

Measurement of GLUT1: Approximately 2×10³ cells were seeded in a 96 well clear poly L lysine (10 μg/cm2) coated polystyrene plate and allow to grow with a media change after every 48 h. on day 8, cells were treated with the optimum concentration of W. Coagulans, Coagulansin-A, insulin, metformin, glibenclamide, and pioglitazone in DMEM:F12 medium supplemented with 2% horse serum for 24 hours. After 24 hours, GLUT1 was measured by using CytoGlow™ cell-based GLUT 1 ELISA kit (Cat no: MBS95002558, MyBioSource, USA), according to the manufacturer's protocol. Briefly, cells were fixed with phosphate-buffered saline (PBS) containing 4% paraformaldehyde at room temperature for 20 mins. Next, cells were quenched for 20 mins at room temperature. Cells were washed in PBS three times, blocked at room temperature for 1 hour. Cells were washed in PBS three times, incubated with primary GLUT1 antibody overnight at 4° C. Cells were washed in PBS three times (200 μl), incubated with HRP-conjugated secondary antibody at room temperature for 1.5 hours. Cells were washed in PBS three times and incubated with a ready-to-use substrate for 30 minutes at room temperature in the dark with gentle shaking on the shaker. The reaction was stopped, and OD was measured at 450 nm immediately using an Emax Plus microplate reader (Molecular devices, CA 94089, USA).

Measurement of Total GLUT4: Approximately 2×10³ cells were seeded in a 96 well clear bottom black poly D lysine (5 μg/cm2) coated plate and allow to grow with a media change after every 48 h. On day 8, cells were treated with the optimum concentration of W. Coagulans, Coagulansin-A, insulin, metformin, glibenclamide, and pioglitazone in DMEM:F12 medium supplemented with 2% horse serum for 24 hours. The supernatant was removed and challenged for 5 mins with 100 nM insulin. Next, cells were washed with warm PBS (37° C.), fixed with phosphate-buffered saline (PBS) containing 4% paraformaldehyde at room temperature for 10 mins. Cells were permeabilized with 0.5% Triton X-100 on PBS for 10 mins at 25° C. The cells were then blocked with 100 μl of 10% NGS in PBS at room temperature for 2 hours. The cells were then incubated with 50 μl GLUT4 antibody (Cat no: NBP1-49533, Novus Biological, USA) diluted 1:100 in block solution overnight at 4° C. Cells were washed in PBS three times (200 μl), incubated with 50 μl Goat anti-Rabbit IgG (H+L) (Cross adsorbed, Alexa Fluor 488, Cat no: R37116, Thermo Fisher Scientific, USA) secondary antibody at room temperature for 30 min. Total fluorescence was measured at 509/617 (excitation/emission) by Tecan multimode plate reader (M200, Tecan, Switzerland). Cells without primary antibody were used for blank reduction.

The relative GLUT 4(%) related to control wells containing cells with DMEM:F12 medium was calculated by (RFU)test/(RFU)control×100. Where (RFU) test is the fluorescence of the test sample and (RFU) control is the fluorescence of the control sample.

Insulin-stimulated 2-NBDG uptake: The ability of the test compounds on insulin-stimulated glucose uptake was measured using the Glucose Uptake Cell-Based Assay Kit (Cat no: 600470, Cayman, Michigan, USA) according to the manufacturer's instructions. Briefly, the cells were seeded at a density of 2×10³ cells/well in a 96 well black, clear-bottom poly D lysine coated plate and allowed to grow with a media change after every 48 hours. On day 8, cells were treated with 100 μl of with the optimum concentration of W. Coagulans, Coagulansin-A, insulin, metformin, glibenclamide, and pioglitazone in DMEM:F12 medium supplemented with 2% horse serum for 24 hours. Subsequently, cells were washed with 300 μl PBS three times and incubated with PBS for 5 mins at 37° C., in a CO₂ incubator. Cells were challenged for 20 min with 50 μl of 100 nM insulin and 50 μl of 100 μg/ml fluorescent glucose analog 2-deoxy-24(7-nitro-2,1,3-benzoxadiazol-4-yl)aminol-D-glucose (2-NBDG) in Krebs/Ringer buffer (KRB) (118.5 mM NaCl, 25 mM NaHCO₃, 4.74 mM KCl, 1.19 mM MgSO₄, 2.54 mM CaCl₂, 10 mM HEPES, 1.19 mM KH₂PO₄, 0.1% BSA) (pH 7.4). At the end of the incubation, the plates were centrifuged for 3 mins at 400×g at room temperature, and the supernatant was aspirated. Cells were washed with 200 μl/well cell-based assay buffer and centrifuged for 3 mins at 400×g at room temperature. The supernatant was aspirated, and cell-based assay buffer was added to each well (100 μl/well). The fluorescence reading of each well was immediately measured (excitation/emission=485 nm/535 nm) by using a Tecan multimode plate reader (Cat no: M200, Tecan, Switzerland). Wells without cells were used for blank reduction. Data were presented as a percentage of diabetic control (untreated cells).

Statistical analysis: One-way ANOVA with Fisher's LSD post hoc test was applied and significance was set at *P<0.05, **P<0.01, and ***P<0.001 by using IBM SPSS to analyze the data. All graphs were generated by using Microsoft Excel.

### Results and Discussion

### Effect of W. Coagulans and Coagulansin-A on Cell Viability of Human Type 2 Diabetes Diseased Skeletal Muscle Myoblasts (D-HSMM)

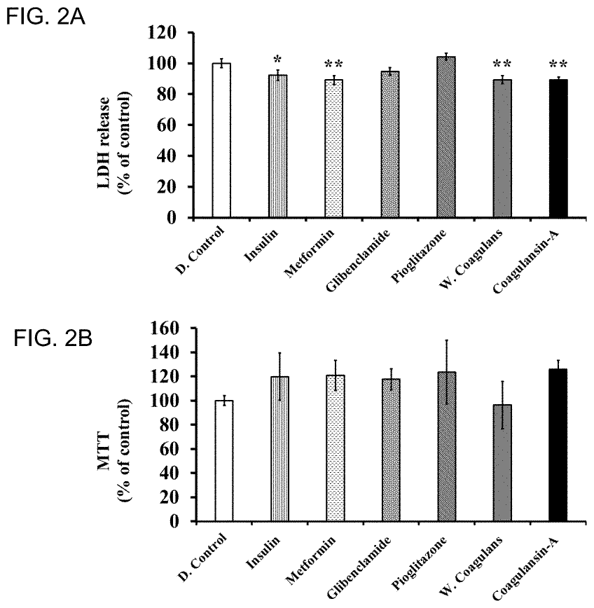

MTT and LDH assays were performed to investigate the effects of Withania Coagulans and Coagulansin-A on cell viability. To determine the effect on LDH release and metabolic rate of D-HSMM, the cells were pretreated with selected concentrations of positive controls, W. Coagulans and Coagulansin-A, and LDH and MTT assays performed (FIGS. 2A, 2B). Results from LDH assay showed a significant decrease in LDH release in Insulin treated cells (p<0.05), Metformin treated cells (p<0.01), W. Coagulans treated cells (p<0.01), and Coagulansin-A (p<0.01) treated cells as compared to control. Results from the MTT assay confirmed that the concentrations of positive controls, W. Coagulans and Coagulansin-A used for treatment in D-HSMM did not affect the viability as compared to the diabetic control (non-treated cells).

The most common assays to determine the safety of novel therapy and to assess its effect on cell viability are LDH and MTT assay. The ease, reliability, and pace are some of the prominent features of these assays. The LDH assay determines membrane integrity by measuring the leakage of the cytosolic enzyme, lactate dehydrogenase. While the MTT assay quantifies mitochondrial activity. The conversion of water soluble MTT (3-[4,5-dimethylthiazol-2-yl]-2,5-diphenyltetrazolium bromide) to an insoluble purple formazan by the action of mitochondrial enzyme succinate dehydrogenase provides a measure of mitochondrial function. The data in the present studies show that W. Coagulans and Coagulansin-A significantly reduced the cytotoxicity of D-HSMM as compared to non-treated control, while they showed a non-significant difference with D-HSMM control in the quantification of metabolic activity through MTT assay.

### Effect of W. Coagulans and Coagulansin-A on Myogenic Regulatory Factors

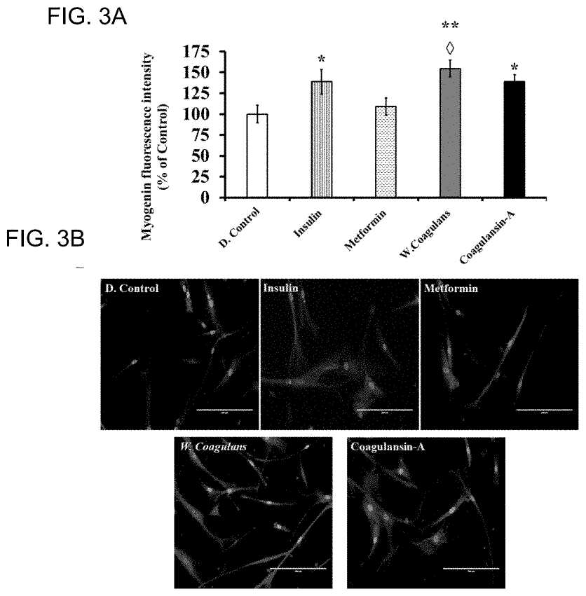

To evaluate the effects of W. Coagulans and Coagulansin-A on diabetes-induced atrophy of D-HSMM myoblasts, immunofluorescence staining of myogenic regulatory factors myogenin and MAFbx/atrogin-1 were performed. As shown in (FIG. 3A), a significantly increased expression of myogenin was observed in Insulin treated cells (p<0.05), W. Coagulans treated cells (p<0.01) and Coagulansin-A treated cells (p<0.05) as compared to D-HSMM control (untreated cells). W. Coagulans treated cells showed enhanced expression (p<0.05) of myogenin as compared to the positive control, metformin. While metformin treated cells remained unaffected as compared to D-HSMM control (untreated cells).

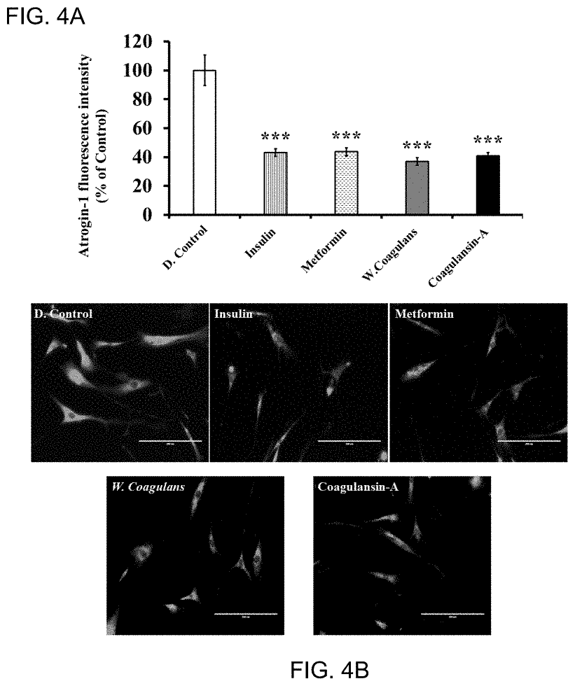

In order to further confirm the protective effects of W. Coagulans and Coagulansin-A on diabetic muscle atrophy, MAFbx/atrogin-1 expression was evaluated in D-HSMM cells. As shown in (FIG. 4A), significantly suppressed expression of atrogin1 was observed in insulin treated cells (p<0.001), metformin treated cells (p<0.001), W. Coagulans treated cells (p<0.001), and Coagulansin-A treated cells (p<0.001).

The orchestrators of myogenesis, the myogenic regulatory factors are crucial for the maintenance of skeletal muscle. The myogenic regulatory factors are the family of transcription factors which contributes to the regulation of muscle protein synthesis and degradation and play a pivotal role in muscle development and regeneration. Myogenin is one of the muscle regulatory factors which is crucial for the initiation of developmental and regenerative myogenesis, as it is involved in myoblast fusion into multinucleated myotubes. On the other hand, in type 2 diabetic condition of muscle-wasting, the transcription of atrogenes that are the markers of the muscle degradation pathway are upregulated, specifically atrogin1/FBXO32. The present data shows that W. Coagulans and Coagulansin-A have the potential to upregulate myogenin, the positive regulator of muscle regeneration. W. Coagulans and Coagulansin-A inhibited the transcription of atrophic marker atrogin1 involved in the upregulation of proteolytic pathways in skeletal muscles.

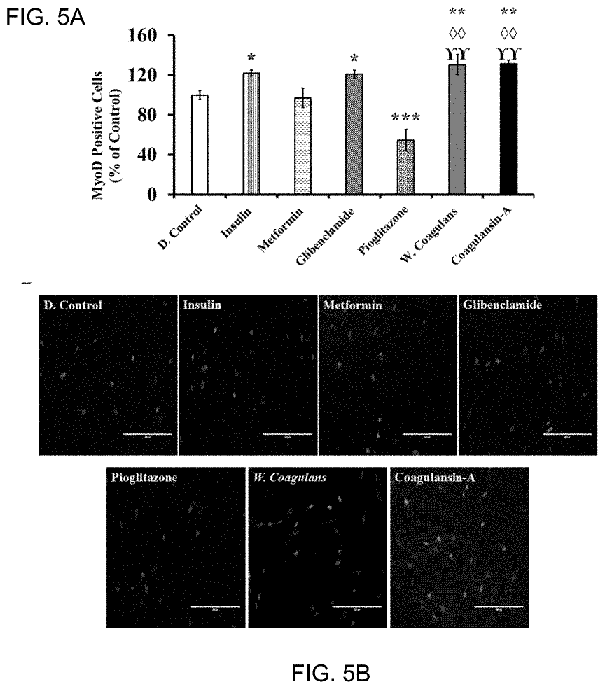

The effect of W. Coagulans and Coagulansin-A was evaluated for MyoD, one of the most important myogenic regulatory factors and muscle specific enhancer for the expression of major glucose transporter GLUT4. An enhanced expression of MyoD positive cells was observed in insulin treated cells (p<0.05), glibenclamide treated cells (p<0.05), W. Coagulans treated cells (p<0.01) and Coagulansin-A treated cells (p<0.01), while pioglitazone treated cells (p<0.001) showed significantly decreased expression as compared to D-HSMM control (non-treated cells). Significantly increased MyoD positive cells were observed in W. Coagulans (p<0.01) and Coagulansin-A (p<0.01) treated myoblasts as compared to metformin and pioglitazone respectively. While metformin treated myoblasts did not show any difference with respect to D-HSMM control (non-treated cells) (FIG. 5A).

### Effect of W. Coagulans and Coagulansin-A Regulation of GLUT4 Expression

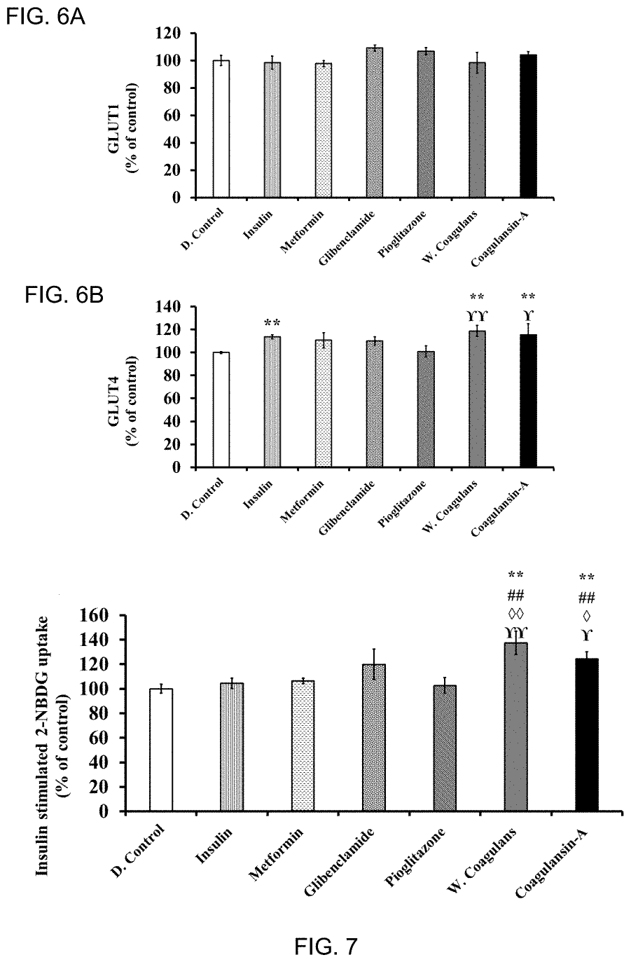

To further investigate the role of W. Coagulans and Coagulansin-A on skeletal muscles insulin sensitivity, the expression of basal non-insulin-dependent glucose transporter GLUT1 and insulin-dependent glucose transporter GLUT4 were evaluated. As shown in (FIG. 6A), the expression of GLUT1 remained unaffected in all the positive controls, W. Coagulans and Coagulansin-A treated D-HSMM myotubes. The results revealed as shown in (FIG. 6B), that the expression of GLUT-4 in the myotubes was upregulated in Insulin treated cells, W. Coagulans treated cells (p<0.01) and Coagulansin-A treated cells (p<0.01) as compared to D-HSMM control (untreated cell). A significant increase was observed in W. Coagulans treated cells (p<0.01) and Coagulansin-A treated cells (p<0.05) as compared to metformin.

The regulation of GLUT4 expression in skeletal muscles is a key to whole-body metabolic homeostasis. The GLUT4 gene transcription is dependent on the binding of myocyte enhancer factor (MEF2) at the 5′-flanking region of 1154 bp. MyoD is not only one of the most important myogenic regulatory factors but is also involved in maintaining the myocyte enhancer activity. MyoD, together with MEF2 induces the expression of the GLUT4 gene in skeletal muscles responsible for the insulin-stimulated glucose uptake. These data suggest that W. Coagulans and Coagulansin-A are potent stimulators of GLUT4 expression through enhancing MyoD in D-HSMM.

### Effect of W. Coagulans and Coagulansin-A on Glucose Uptake.

As skeletal muscle is a principal site for glucose disposal, the effect of W. Coagulans and Coagulansin-A was evaluated on insulin-stimulated glucose uptake in D-HSMM myotubes. As shown in (FIG. 7A), significantly enhanced glucose uptake was observed in W. Coagulans treated myotubes (p<0.01) and Coagulansin-A (p<0.01) treated myotubes as compared to control (untreated cells). W. Coagulans treated myotubes (p<0.01) and Coagulansin-A treated myotubes (p<0.01), also showed increased glucose uptake as compared to positive controls, insulin, metformin, and pioglitazone. While the positive control treated cells remained unaffected in glucose uptake as compared to D-HSMM control (untreated cells).

Glucose uptake in skeletal muscles is mainly regulated by insulin signal transduction, GLUT4 expression, and translocation. In type 2 diabetes due to insulin resistance, impairment in the insulin signaling pathway, suppressed GLUT4 transcription, the regulation of glucose uptake is altered. It is evident from the results in the present study that W. Coagulans and Coagulansin-A efficiently improved the insulin-stimulated glucose uptake in D-HSMM.

Several studies showed the beneficial effect of herbal medicines in type 2 diabetes mellitus due to the presence of multiple pharmacologically active compounds. The present study successfully demonstrated the positive in vitro effects of W. Coagulans and Coagulansin-A on myogenesis regulation, GLUT4 expression, and glucose uptake in human type 2 diabetic skeletal muscle myoblasts.

### W. Coagulans and Coagulansin-A Dose Optimization.

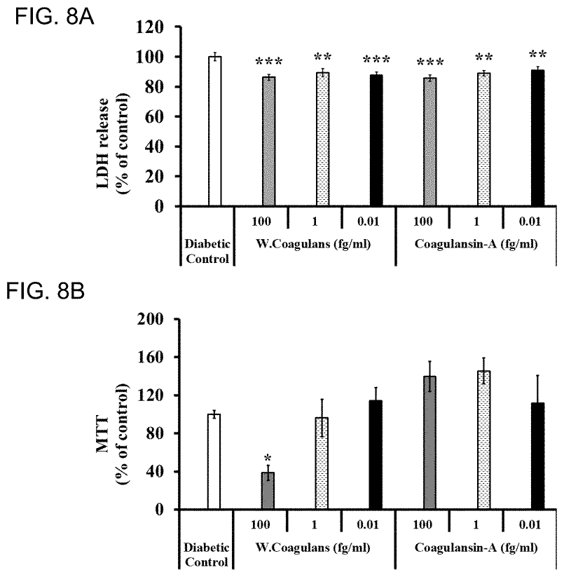

To determine the nontoxic optimal dose of W. Coagulans and Coagulansin-A, MTT and LDH assays were performed to investigate their effect on cell viability and metabolic rate of D-HSMM. The cells were pretreated with 10⁻¹³ g/ml, 10⁻¹⁵ g/ml, and 10⁻¹⁷ g/ml concentrations of W. Coagulans and Coagulansin-A and performed the LDH and MTT assay respectively as depicted in (FIGS. 8A-B).

Results from LDH assay showed a significant decrease in LDH release in all the concentrations of W. Coagulans treated cells, and Coagulansin-A treated cells as compared to untreated diabetic control. Results from the MTT assay confirmed that all the concentrations of W. Coagulans and Coagulansin-A used for treatment in D-HSMM did not affect the metabolic rate, except 10⁻¹³ g/ml concentration of W. Coagulans as compared to the control (non-treated cells).

In order to further confirm the nontoxic optimal concentration of W. Coagulans and Coagulansin-A on DSMM, we evaluated myoD, the myogenic regulatory factor and muscle specific enhancer for the expression of GLUT4 by using 10⁻¹³ g/ml, 10⁻¹⁵ g/ml, and 10⁻¹⁷ g/ml concentrations of W. Coagulans and Coagulansin-A as shown in (FIG. 9). An enhanced expression of MyoD positive cells was observed for the concentration 10⁻¹⁵ g/ml for both W. Coagulans treated cells (p<0.01) and Coagulansin-A treated cells (p<0.01) as compared to diabetic control.

Previous studies demonstrated detailed mechanism and models for insulin resistance in skeletal muscles, as they are the fundamental site of metabolic balance in body. The skeletal muscle insulin resistance activates proteolytic signaling pathways which lead to the condition of muscle atrophy. Skeletal muscle is a major factor to determine the quality of life through regulation of glucose homeostasis, muscle strength to perform the physical function, and capacity to recover from illness. Altogether, skeletal muscle is one of the most important tissues, affected severely due to insulin resistance. Several drugs currently in use to improve insulin action, which may include the skeletal muscle, but none specifically acts to improve muscle metabolic homeostasis and attenuate muscle atrophy.

The anti-diabetic activity of Coagulansin-A has potential to regain metabolic homeostasis in skeletal muscles of diabetic patients. Coagulansin-A is a steroidal lactone isolated from the methanolic fraction of W. Coagulans.

Coagulansin-A has antiurease activity and has beneficial effects on development of bovine embryo. Previous studies have proved the promising euglycemic effect of W. Coagulans, which may involve multiple mechanisms.

### Example 2: Withania Coagulans Revives Functional Beta Cells Mass in Chronic Diabetic Rats by Attenuating AGE-RAGE Mediated Oxidative Stress

The role of advanced glycation end-products (AGEs), the AGE receptor (RAGE), oxidative stress, and inflammation in beta-cells degeneration is well documented. Although previous studies have provided evidence of mechanism involved in pancreatic beta cells demise, none of these reported to stimulate the regeneration of functional beta cells mass in chronic diabetes. This Example is the first to report the therapeutic potential of Withania Coagulans to improve pancreatic beta cells mass and function by attenuating AGEs-RAGE induced oxidative stress. These results demonstrate the potential of Coagulansin A as a breakthrough therapy for beta cells regeneration in type 2 diabetes mellitus, as beta cells revival should be the putative target at each intervention stage of Diabetes mellitus.

### Methods

Preparation of extract: The shade dried fruits of Withania Coagulans were crushed to a coarse powder by using a grinder (Moulinex, France) and then soaked in distilled water overnight followed by filtration through the cheesecloth. The dose of 10 mg/kg body weight is selected concerning the initial screening of aqueous extract through dose-response studies previously performed in our laboratory.

Animals: Male Wister rats weighing (225-250 g) were obtained from the certified Animal Research Facility of College of Medicine and Health Sciences, United Arab Emirates University, Al Ain, UAE. The rats (3 rats/cage) were housed in a pathogen-free controlled environment at 23±2° C. in a 12-hour light/dark cycle with food and water ad libitum. All the experimental procedures were approved by the United Arab Emirates University Animal Ethics Committee and carried out in accordance with laboratory animal care and use guidelines by the National Academy of Science.

Experimental Diabetes was induced by the single intraperitoneal injection of Streptozotocin (STZ) (60 mg/kg) dissolved in 0.1M fresh ice-cold citrate buffer at pH 4.5 immediately before use. The age match control received citrate buffer only. After 48 hours of STZ injection, postprandial blood glucose was measured using ACCU-CHEK Performa Glucometer (Roche Diagnostics Australia CASTLE HILL NSW 1765) and rats with blood glucose level >250 mg/dL were considered diabetic. After diabetes induction, rats were kept under standard animal laboratory practices for eight months and carefully monitored for use in present studies.

Experimental Design: Eight months post-STZ injection, the rats with postprandial blood glucose levels in the range of 250-350 mg/dl were considered as chronic type II diabetic and deemed suitable for this study. The rats were divided into four respective groups each containing eight rats for the long-term treatment phase, Non-Diabetic Control, Non-Diabetic Withania Coagulans treated, Diabetic Control, and Diabetic Withania Coagulans Treated. The treated groups received the 10 mg/kg of Withania Coagulans aqueous fruit extract, and the Control groups received the same dose of distilled water through oral gavage for 40 days.

Fasting blood glucose: Post 40 days of treatment of Withania Coagulans, a fasting blood sample was taken from all the groups included in this study. After 8 hours fast, the blood glucose levels of the rats were monitored through the tail vein using ACCU-CHEK Performa Glucometer (Roche Diagnostics, Australia)

Sample collection and Tissue Homogenate preparation: Rats were sacrificed through decapitation after the completion of Withania Coagulans aqueous fruit extract forty days treatment and the blood samples were collected for analysis of biochemical parameters. The pancreas was removed and washed in ice-cold phosphate buffer saline (PBS), and immediately frozen in liquid nitrogen and stored at −80° C. For proteomic analysis, the frozen pancreas was cut into small pieces with the help of a sharp blade, weighed, and homogenized with ten 2.8 mm ceramic beads (Omni International, USA) in cryotubes (Omni International, USA) by using Bead Ruptor 4 (Omni international) at speed 5 for 30 seconds. The tissues were homogenized with 10 volumes of ice-cold KCl lysis buffer (10 mM Tris-HCl, pH 8.0, 140 mM NaCl, 300 mM KCl, 1 mM EDTA, 0.5% v/v Triton X-100 and 0.5% w/v sodium deoxycholate) with protease and phosphatase inhibitor cocktails (Thermo Scientific, USA). The resulting homogenates, after 30 min incubation on ice, were centrifuged (Eppendorf centrifuge, USA) at 15,000 rpm for 30 min at 4° C. The resulting supernatant was pancreatic homogenate, which was stored at −80° C. until further biochemical analysis.

The Enzyme-Linked Immunosorbent Assay (ELISA): Post-treatment insulin level was examined by using the High Range Rat Insulin ELISA kit (Mercodia, Uppsala, Sweden), which could detect the trace insulin as low as 1.74 pmol/1 in serum, in accordance with the manufacturer's protocol. The serum Glucagon (DY1249) was assessed using commercially available R&D sandwich ELISA kits and expressed as pg/ml. For the quantitative detection of serum AGEs and pancreatic AGEs, competitive ELISA kits (ab238539) were used (Abcam, USA). The concentration of AGEs is expressed μg per mg of protein, respectively. The pancreatic IAPP were estimated by using the commercially available pre-coated competitive ELISA kit (MB S2700315) in rat pancreatic homogenate (MyBioSource, San Diego, CA, USA).

The cytokine CRP (DY1744) and Rage (DY1616) protein levels in the pancreas were quantitated using commercially available ELISA kits (DuoSet ELISA, R&D Systems, Minneapolis, USA) with minor modifications. The assay was carried out by coating the Nunc MaxiSorp 96-well plate (ThermoFisherScientific, USA) with capture antibody working concentration in PBS, sealed and incubated overnight followed by incubating the plate at room temperature with kit specified Block buffer. The respective standards, blank, and pancreatic homogenate samples were added, and the plate was incubated overnight at 4° C., followed by incubating the plate with the working concentration of detection antibody, Streptavidin-HRP, TMB substrate with kit specified incubation time. Proper washing of plate was conducted with a kit specified wash buffer to achieve the proper result by using a plate washer (Hydroflex microplate washer, Tecan, Switzerland). Stop solution was added to each well and color intensity was measured spectrophotometrically by reading absorbance at 450 nm using Emax Plus microplate reader (Molecular devices, CA 94089, USA). The concentration of CRP and Rage is expressed in the pancreas as pg/mg of protein.

Oxidative stress markers: The levels of oxidative stress markers were determined according to the manufacturer's protocol using commercially available kits.

The level of total Glutathione (GSSH+GSH) was estimated following the protocol of assay kit (Sigma Chemical Co., St. Louis, MO, USA) in the pancreatic homogenate. For the measurement of total Glutathione, the pancreatic homogenate was deproteinized by mixing equal volumes of protein homogenate and 5% (w/v) sulfosalicylic acid (SSA) solution, vortexed (Vortex-Genie 2, scientific industries, USA) immediately, and then incubated on ice for 10 mins following centrifugation (Eppendorf centrifuge, USA) at 10,000×g for 10 min. The supernatant was collected to determine the total Glutathione activity in samples. The Glutathione assay kit uses the kinetic assay through the action of catalytic amounts (nano moles) of GSH to form a yellow-colored product (5-thio-2-TNB) which was measured spectrophotometrically against a blank at 405 nm within 5 mins by using Emax Plus microplate reader (Molecular devices, CA 94089, USA). GSH concentration was expressed as μM.

The activity of the Catalase enzyme was assessed in pancreatic homogenate by using the Catalase assay kit (Cayman Chemical Company, Ann Arbor, MI, USA). This method utilizes the peroxidative function of the catalase enzyme with methanol to produce formaldehyde in the presence of H₂O₂. The formaldehyde reacts with chromogen (4-amino-3-hydrazino-5-mercapto-1,2,4-trizazole) to form a colored product which was measured spectrophotometrically at 540 nm by using Emax Plus microplate reader (Molecular devices, CA 94089, USA). The catalase enzyme activity in the pancreatic homogenate was expressed as nmol/min/mg tissue.

The metalloenzyme Superoxide Dismutase (SOD) activity was assessed in pancreatic homogenate by using a SOD assay kit (Cayman Chemical Company, Ann Arbor, MI, USA). This colorimetric assay was performed according to the manufacturer's protocol which utilizes the tetrazolium salt for the detection of superoxide radicals generated by xanthine oxidase and hypoxanthine. The activity of SOD enzyme was measured spectrophotometrically at an absorbance of 450 nm by using Emax Plus microplate reader (Molecular devices, CA 94089, USA) and expressed in units (U) per mg of protein.

Cayman's Nitrate/Nitrite colorimetric assay kit was used to analyze the Nitric Oxide (NO) level in the pancreatic homogenate. This kit protocol by utilizing the nitrite reductase and Griess reagents with required incubation time in simple two steps to produce a purple azo compound which was measured spectrophotometrically at an absorbance of 540 nm using Emax Plus microplate reader (Molecular devices, CA 94089, USA). The concentration of NO in tissue homogenate was expressed as μM per milligram of protein.

Malondialdehyde (MDA), a product of lipid peroxidation which is a marker for cellular injury was measured in the pancreatic homogenate using the TBARS Assay kit (Cayman Chemical Company, Ann Arbor, MI, USA). The TBARS assay measures the MDA-TBA adduct formed as a result of the reaction between MDA from sample and TBA under a specified high-temperature acidic environment (90-100° C.). The resultant colored adduct was measured spectrophotometrically at an absorbance at 540 nm using Emax Plus microplate reader (Molecular devices, CA 94089, USA). The concentration of MDA in a sample was expressed as μM per milligram of protein.

Immunohistochemical Analysis: The pancreatic tissue was fixed in 4% formaldehyde, then it was embedded in paraffin and sectioned at 4 μm thickness. The tissue slices were fixed on microscope slides and the paraffin was eliminated with a xylene rinse. The slices were dehydrated by washing twice with absolute alcohol, then rinsed with 95%, 70%, and 50% ethanol respectively for 3 minutes each, followed by washing with distilled water for 5 minutes. After antigen retrieval, sections were immunoreacted with respective primary antibodies (Abs) overnight at 4° C. The tissue section was placed in a protein block (X0909) for 45 minutes at room temperature (Dako, Carpinteria, CA, USA). For immunohistochemical analysis, IAPP and insulin in the pancreas islets were detected using a rabbit monoclonal antibody (SAB4200493) diluted 1:500 (Sigma-Aldrich, St Louis, MO, USA), and Guinea pig polyclonal antibody (A0564) diluted 1:500 (Dako, Carpinteria, CA, USA). The procedure is followed by immunofluorescence staining with Alexa Fluor® 488 labeled anti-rabbit (ab181448) diluted 1:10 (Abcam, Cambridge, MA, USA) and Alexa Fluor® 594 labeled anti-guinea pig (106-585-006) diluted 1:100 (Jackson, Cambridgeshire, UK) and mounted with Immuno mount. Images were obtained with a microscope (Fluorescent and Confocal Microscope, Nikon Co. Tokyo, Japan) at 60×oil objective magnification using identical acquisition settings for each section.

The analyze the islet dimensions and the insulin-positive immunoreactive pancreatic beta-cells, above protocol mentioned, was repeated for insulin immunofluorescence analysis in the pancreas islets by using a Guinea pig polyclonal antibody (A0564) diluted 1:500 (Dako, Carpinteria, CA, USA) followed by Alexa Fluor® 594 labeled anti-guinea pig (106-585-006) diluted 1:100 (Jackson, Cambridgeshire, UK). The nuclei were labeled using DAPI (Hoechst 33342) staining (Thermo Fisher Scientific Inc, Massachusetts, U.S.A.). Images were obtained with a microscope (EVOS FLoid Cell Imaging Station, Thermo Fisher Scientific, U.S.A.) at a magnification of 20×using identical acquisition settings for each section.

All the images were processed by the Fiji/ImageJ program (http://rsbweb.nih.gov/ij/) and, then recorded and compared statistically.

Statistical analysis: One-way ANOVA with an LSD post hoc test was applied and significance was set at *P<0.05, **P<0.01, and ***P<0.001 by using IBM SPSS to analyze the data. All graphs were generated by using Microsoft Excel 2013.

### Results

Attenuation of Blood Glucose, Serum Insulin, and Serum Glucagon Levels after Withania Coagulans Administration

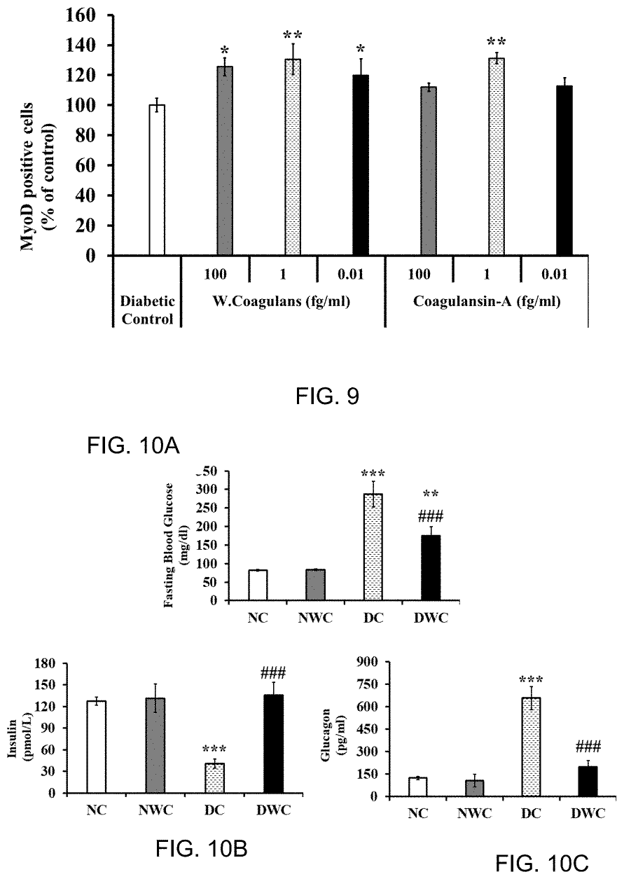

FIGS. 10A-C show the effect of Withania Coagulans-treatment on streptozotocin-induced eight months chronic diabetic rats. A significant attenuation (p<0.001) of fasting blood glucose levels in the diabetic Withania Coagulans treated group after 40 days of treatment was observed when compared to the diabetic control group (FIG. 10A). After 40 days of treatment, serum insulin level was increased (p<0.001) in the diabetic Withania Coagulans treated group (FIG. 10B). Similarly, increased serum glucagon level was significantly decreased (p<0.001) in the diabetic Withania Coagulans treated group (FIG. 10C).

STZ-induced diabetes mimics the metabolic abnormalities and beta cells demise related to diabetes mellitus. The cytotoxicity of STZ in beta cells is accompanied by low insulin and high glucagon that led to high blood glucose levels. The persistent hyperglycemia leads to the production and accumulation of AGEs in pancreatic beta cells. AGEs by their interaction with Rage enhances oxidative stress and further exacerbates the beta cell damage. The present study revealed the effect of forty days oral administration of Withania Coagulans aqueous fruit extract exerted a significant anti-diabetic effect on chronic eight-month STZ-induced diabetic rats. The increased plasma glucose, enhanced serum AGEs, decreased serum insulin level and elevated glucagon level are significantly attenuated by Withania Coagulans.

Improvement of Serum AGEs Levels, Pancreatic AGEs, and RAGE Expression and Normalization of Pancreatic IAPP after Withania Coagulans Administration

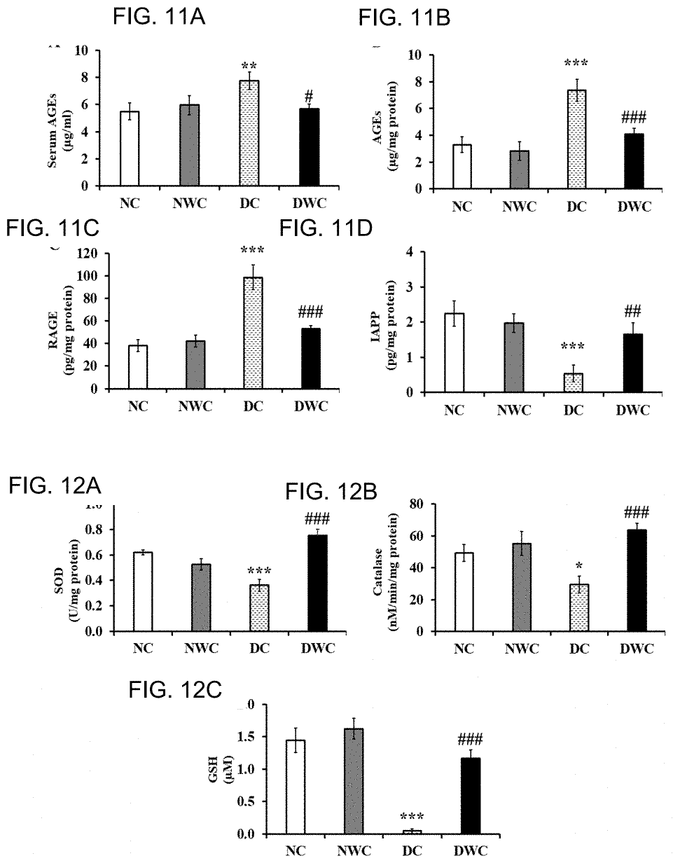

The level of serum AGEs was significantly high (p<0.01) in streptozotocin-induced diabetic control rats as compared to Non-diabetic control. The Withania Coagulans treated group showed a significant (p<0.05) decrease in serum AGEs level when compared to the diabetic control group (FIG. 11A). A significant increase (p<0.001) in the pancreatic AGEs was observed in the diabetic control group compared to the non-diabetic control group. However, the administration of Withania Coagulans in the diabetic treated group significantly (p<0.001) suppressed pancreatic AGEs accumulation compared to the diabetic control group (FIG. 11B). The pancreatic RAGE expression was significantly (p<0.001) enhanced in the diabetic control group when compared to the non-diabetic control group. Administration of Withania Coagulans showed a significant (p<0.001) decrease in pancreatic RAGE expression level compared to the diabetic control group (FIG. 11C). The pancreatic IAPP level was significantly (p<0.001) declined in the diabetic control group which was significantly (p<0.01) normalized after Withania Coagulans treatment when compared to the diabetic control group (FIG. 11D).

AGEs-RAGE interaction activates various signaling pathways which induce inflammation and increases ROS level through oxidative stress. Recent studies suggest that the blockade or deletion of RAGE attenuated the oxidative stress contributing to pancreatic beta cells inflammation, toxicity, and apoptosis. In the present study, administration of Withania Coagulans reduced the pancreatic AGEs and downregulated RAGE expression levels. These results seem to elucidate the protective effect of Withania Coagulans on AGE-induced pancreatic damage.

The IAPP that is co-localized and co-secreted with insulin plays a substantial role in maintaining normoglycemia by suppression of glucagon release and regulation of gastric emptying. Unlike human IAPP (hIAPP), rat IAPP (rIAPP) is non-toxic due to the structural difference in the amyloidogenic region of the peptide. The rIAPP contains three proline residues at 25, 28, and 29 positions which contribute to its ability to prevent the formation of pathogenic IAPP aggregates. Early studies have shown that the expression level of IAPP was also reduced parallel to insulin in chronic STZ-induced diabetes mellitus. The IAPP-deficient mice displayed more severe beta cells degeneration in alloxan-induced diabetes mellitus which explains the protective role of IAPP in the rodent pancreas. Administration of Withania Coagulans showed a significant increase in pancreatic IAPP levels as compared to the diabetic control group.

### Protective Effects of Withania Coagulans Administration on Pancreatic Oxidative Stress Markers SOD, Catalase, and GSH Levels

The pancreatic homogenate of chronic eight-month diabetic rats showed critically depleted activity of these antioxidant enzymes that explains the increased cellular ROS production. The SOD activity was significantly (p<0.001) reduced in eight months old chronic diabetic control group as compared to the non-diabetic controls. However, the SOD activity was significantly (p<0.001) increased in Withania Coagulans treated diabetic group (FIG. 12A). Pancreatic Catalase activity was significantly (p<0.05) suppressed in the diabetic control group compared to the Non-diabetic control group. Treatment with Withania Coagulans showed a significant (p<0.001) increase in pancreatic catalase activity when compared to the diabetic control group (FIG. 12B). The GSH level was significantly (p<0.001) reduced in the pancreas of eight months old chronic diabetic control group when compared to the non-diabetic control group. However, the administration of Withania Coagulans significantly (p<0.001) increased the pancreatic GSH level when compared to the diabetic control group (FIG. 12C).

The improved level of SOD, catalase, and glutathione in Withania Coagulans treated diabetic group suggests that Withania Coagulans tends to have free radical scavenging activity and, by upregulation of antioxidant enzymes, Withania Coagulans protect the beta cells from oxidative stress damage.

Oxidative stress has a pivotal role in the mechanism underlying pancreatic beta cells degeneration. The enhanced production of free radicals without an efficient cell defense mechanism leads to lipid peroxidation which may bring further cellular damage. The function of antioxidant enzymes SOD, catalase, and glutathione is to protect the cell against free radical damage. SOD detoxifies the cell by catalyzing the superoxide free radical to generate hydrogen peroxide and molecular oxygen. The catalase and glutathione neutralize the enhanced hydrogen peroxide production. The decrease in the expression and activity of SOD, catalase, and glutathione in diabetes mellitus could be the result of hydrogen peroxide accumulation, enzymes inactivation by glycosylation, or increased deprivation of antioxidant enzymes due to enhanced oxidative stress that surpasses the cellular antioxidant capacity. The pancreatic homogenate of eight-month chronic diabetic rats showed critically depleted activity of these antioxidant enzymes that explains the increased cellular ROS production. The level of SOD, catalase, and glutathione significantly improved in the Withania Coagulans treated diabetic group. These results suggest that Withania Coagulans mediates free radical scavenging activity by upregulation of antioxidant enzymes that protect the beta cells from oxidative stress damage.

Mitigation of Pancreatic MDA, NO, and CRP after Withania Coagulans Administration

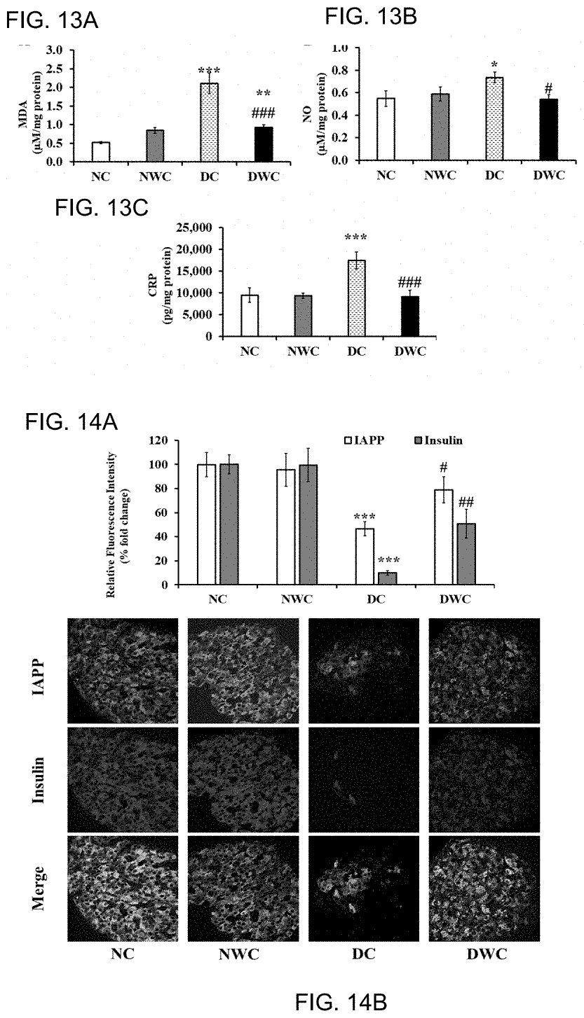

The increased MDA level group is the consequence of lipid peroxidation due to reduced antioxidants and enhanced ROS. The pancreatic lipid peroxidation was significantly (p<0.001) elevated in chronic diabetic control group compared to the non-diabetic control group and Withania Coagulans administration showed a significant (p<0.001) reduction of pancreatic MDA level compared to the Diabetic control group (FIG. 13A).

The STZ is nitric oxide (NO) donor. Previously performed studies showed that STZ-induced beta-cell toxicity and DNA damage are mediated by NO expression. An increase in NO level in the pancreas of STZ-induced chronic eight months diabetic rats was observed, and increased (p<0.05) pancreatic NO level in the diabetic control group was significantly (p<0.05) decreased after the administration of Withania Coagulans aqueous extract (FIG. 13B).

The hyperglycemia-induced oxidative stress and lipid peroxidation enhance inflammation that induces beta-cell apoptosis. Previous studies reveal that increased CRP, an important biomarker of inflammation signifies islet cell destruction in rats. Pancreatic CRP levels were significantly (p<0.001) increased in the diabetic control group when compared to the non-diabetic control group, however, this increase in CRP level was significantly (p<0.001) attenuated in the Withania Coagulans treated diabetic group (FIG. 13C).

The increased MDA level is the consequence of lipid peroxidation due to reduced antioxidants and enhanced ROS. A significant increase in MDA levels in the pancreas of chronic eight-month STZ-induced diabetic control was observed in this study. Administration of Withania Coagulans reduced the MDA level in the pancreatic homogenate of the treated group when compared with the diabetic control group. These results further confirm that Withania Coagulans exhibits free radical scavenging properties. The STZ is a nitric oxide (NO) donor. Previously performed studies showed that STZ-induced beta cells toxicity and DNA damage are mediated by NO expression. An increase in NO level was observed in the pancreas of eight-month chronic STZ-induced diabetic control. However, Withania Coagulans significantly reduced the increased NO production. The hyperglycemia-induced oxidative stress and lipid peroxidation enhance inflammation that induces beta cells apoptosis. Previous studies reveal that increased CRP, an important biomarker of inflammation signifies islet cell destruction in rats. In the pancreas of STZ-induced chronic eight-month diabetic rats, an increased level of CRP was observed which was significantly attenuated after Withania Coagulans treatment.

Immunofluorescence Analysis of IAPP and Insulin in Pancreatic Beta Cells after Withania Coagulans Administration

To further evaluate the effect of Withania Coagulans on pancreatic beta cells of STZ-induced chronic eight-months diabetic rats, IAPP, and insulin double-immunofluorescence staining of the pancreatic tissue sections was examined. The present studies confirm IAPP and insulin are colocalized in pancreatic beta-cells. The pancreatic islets from the Diabetic control group showed a significant decrease (p<0.001), (p<0.001) in IAPP and insulin immunoreactivity respectively (FIG. 14A). Administration of Withania Coagulans showed a significant increase in (p<0.01) in pancreatic insulin staining and subsequently showed a significant revival in pancreatic IAPP staining as compared to the Diabetic control group (FIG. 14A).

To evaluate the abnormalities in pancreatic beta cell histology of STZ-induced eight-months chronic diabetic rats, insulin and IAPP immunofluorescence in pancreatic islets was analyzed. Shrinkage of pancreatic islets, a decrease in the beta cells mass, and a reduced number of islets was observed in the pancreas of the STZ-induced eight-months chronic diabetic control group. However, oral administration of Withania Coagulans appeared to preserve and recover beta cells mass and showed a protective effect on pancreatic beta cells integrity. The diabetic treated group showed an increased percent of beta cells by higher insulin immunofluorescence staining, improved pancreatic beta cell mass, and a significant increase in pancreatic islets number. The Withania Coagulans treatment showed a parallel increase in IAPP immunofluorescence as that of insulin immunofluorescence in the pancreas of STZ-induced chronic eight-months diabetic rats.

Improvement of Pancreatic Beta Cells Growth and Preservation of Islet Viability after Withania Coagulans Administration

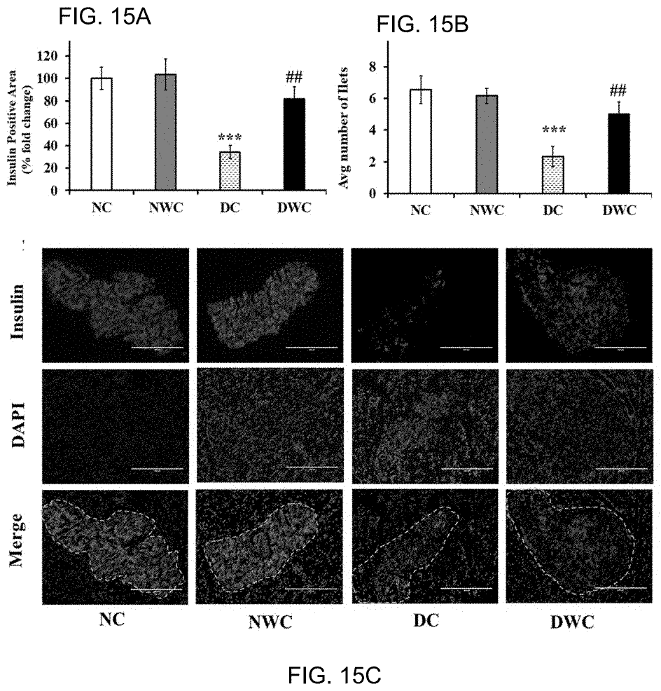

The immunofluorescence studies confirm that the pancreatic islet area as well as the insulin-positive beta-cells were reduced (p<0.001) in streptozotocin-induced eight months chronic diabetic rats (FIG. 15A). The islet cells were almost lost and there was a significant decrease observed in the number of pancreatic islets per sample (p<0.001) as compared to the non-diabetic control group (FIG. 15B). However, the Withania Coagulans administration showed regeneration of the insulin-positive beta-cells (p<0.01) and also significantly increased (p<0.01) the number of pancreatic islets per sample as compared to the diabetic control group.

Without being bound by theory, there is evidence that pancreatic beta cells can regenerate from pre-existing beta cells provided with extra pancreatic stimulators. The present example demonstrates the regenerative action of Withania Coagulans on pancreatic beta cells is mediated by increased beta cells density and enhanced volume of islets in the pancreas of STZ-induced diabetic rats. These findings elucidate the therapeutic potential of Withania Coagulans in the management of diabetes by capitalizing on the ability of the aqueous fruit extract to alleviate the degenerative effects of STZ in the pancreatic tissue

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

All publications and patent literature cited in this specification are herein incorporated by reference as if each individual publication or patent were specifically and individually indicated to be incorporated by reference. The publication or patent literature are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

It is noted that, as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Other embodiments of the present disclosure are possible. Although the description above contains specific details, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus, the scope of this disclosure should be determined by the appended claims and their legal equivalents.

What is claimed is:

1. A composition for treatment of Type 2 diabetes, the composition comprising a therapeutically effective amount of Coagulansin A and a pharmaceutically or nutraceutically acceptable carrier, wherein the therapeutically effective amount is effective for treating muscle atrophy, treating insulin resistance in skeletal muscle, initiating muscle regeneration, upregulating myogenic regulatory factors, repressing atrogin-1 expression, improving insulin-stimulated glucose uptake, inhibiting advanced glycation end-products (AGEs)-mediated β-cell death, increasing β-cell density, enhancing pancreatic islets volume, upregulating insulin-dependent glucose transporter GLUT4 expression in skeletal muscle, or a combination thereof.

2. The composition of claim 1, wherein the composition is formulated for buccal, sublingual, oral, nasal, pulmonary, transdermal, intravenous, intraarterial, intraperitoneal, intramuscular, or subcutaneous administration.

3. The composition of claim 1, wherein the composition is formulated as a capsule, tablet, pill, dragee, powder, granule, emulsion, solution, suspension, syrup, elixir, or implant.

4. The composition of any one of claim 1, wherein the composition is formulated for delayed release, extended release, or immediate release.

5. The composition of any one of claim 1, wherein the composition further comprises insulin, an Alpha-glucosidase inhibitor, a biguanide, a dopamine agonist, a Dipeptidyl peptidase-4 (DPP-4) inhibitor, a Glucagon-like peptide-1 receptor agonist, a meglitinide, a Sodium-glucose transporter (SGLT) 2 inhibitor, a sulfonylurea, a thiazolidinedione, or a combination thereof.

6. A method of treating type 2 diabetes in a subject in need thereof, the method comprising administering a therapeutically effective amount of an aqueous extract of Withania Coagulans fruit, wherein the aqueous extract comprises Coagulansin A, or a derivative or analogue thereof.

7. The method of claim 6, wherein the therapeutically effective amount is effective for treating muscle atrophy, treating insulin resistance in skeletal muscle, initiating muscle regeneration, upregulating myogenic regulatory factors, repressing atrogin-1 expression, improving insulin-stimulated glucose uptake, inhibiting advanced glycation end-products (AGEs)-mediated β-cell death, increasing β-cell density, enhancing pancreatic islets volume, upregulating insulin-dependent glucose transporter GLUT4 expression in skeletal muscle, or a combination thereof.

8. The method of claim 6, wherein the extract is administered in combination with insulin, an Alpha-glucosidase inhibitor, a biguanide, a dopamine agonist, a Dipeptidyl peptidase-4 (DPP-4) inhibitor, a Glucagon-like peptide-1 receptor agonist, a meglitinide, a Sodium-glucose transporter (SGLT) 2 inhibitor, a sulfonylurea, a thiazolidinedione, or a combination thereof.

9. The method of claim 6, wherein the extract is administered via a buccal, sublingual, oral, nasal, pulmonary, transdermal, intravenous, intraarterial, intraperitoneal, intramuscular, or subcutaneous administration route.

10. The method of claim 6, wherein the subject is a mammal.

11. The method of claim 10, wherein the mammal is a human.

12. The method of claim 6, wherein the extract is administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg extract, of subject body weight per unit dose.

13. The method of any one of claim 6, wherein the extract is administered at a dosage level sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg Coagulansin A, of subject body weight per unit dose.

14. The method of any one of claim 6, wherein the extract is administered one or more times a day.

15. The method of claim 6, wherein the extract is administered at least once a day for at least two days.

16. The method of claim 15, wherein the extract is administered for at least one week.

17. The method of claim 15, wherein the extract is administered for at least one month.

18. The method of claim 6, wherein the extract is administered with at least one meal.

19. The method of claim 18, wherein the extract is administered at start of the meal or after the meal.

20. The method of claim 6, wherein the extract is administered as an adjunct to diet and exercise to improve glycemic control.
